(19) United States
(12) Patent Application Publication
(10) Pub. No.: US 2007/0016020 A1
(43) Pub. Date: Jan. 18, 2007

(54) Ultrasonic probe, ultrasonographic device, and ultrasonographic method

(76) Inventors: Mitsuhiro Oshiki, Chiba (JP); Hiroshi Kanda, Chiba (JP); Ryuichi Shinomura, Chiba (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/574,272

(22) PCT Filed: Sep. 24, 2004
(86) PCT No.: PCT/JP04/13949

§ 371 Date: Mar. 31, 2006

(30) Foreign Application Priority Data
Oct. 2, 2003 (JP) 2003-344512

(51) Int. Cl. A61B 8/00

(52) U.S. Cl. 600/437

(57) ABSTRACT

An ultrasonic probe 10 is formed by arranging a plurality of transducers 26a to 26m for converting drive signals into ultrasonic waves to transmit the waves to an object to be inspected, and receiving ultrasonic waves generated from the object to convert the waves into electrical signals. Each of the transducers 26a to 26m has a plurality of oscillation elements 34-1 to 34-30, and each of the oscillation elements 34-1 to 34-30 has a characteristic in which the electromechanical coupling coefficient changes in accordance with the strength of the direct-current bias applied by being superposed on the drive signals. Electrodes 35, 36, and 37 of each of the oscillation elements 34-1 to 34-30 are connected to terminals 49-1 and 49-2 to which the drive signals are applied.

### TECHNICAL FIELD

The present invention relates to an ultrasonic probe for picking up an ultrasound image (for example, a diagnostic image) of an object to be inspected, an ultrasonic imaging apparatus, and an ultrasonic imaging method.

An ultrasonic imaging apparatus transmits and receives ultrasonic beams to and from an object to be inspected by an ultrasonic probe, and reconstructs an ultrasound image based on electrical signals output from the ultrasonic probe. The ultrasonic probe is formed by arranging a plurality of ultrasonic transducers which convert electrical signals into ultrasonic waves and vice versa.

In general, the transducers of this ultrasonic probe are formed by a piezoelectric material such as crystal, piezoelectric ceramics. Thus, the width of each transducer has a relatively large size (for example, a few millimeters) as a result of the manufacturing process, etc., of the piezoelectric material. Accordingly, the mutual distances among the plurality of transducers become large, and a certain limitation arises in the improvement of the resolution (resolving power) of an ultrasound image.

It is therefore desired to improve the resolution by decreasing the width of the transducers in the array direction including the method of manufacturing. Also, it is desired to develop an ultrasonic probe capable of changing the sound pressure of ultrasound beams in accordance with the distance between an imaging portion and the ultrasonic probe.

Also, the resolution of an ultrasound image depends on the beam width or the diameter (in the following, generically called a beam width) at the focal point resulting from the sound-pressure distribution of ultrasound beams. The beam width is determined by the width in the array direction (in the following, called a major-axis direction) of transducers and the width of the orthogonal direction to the major-axis direction (in the following, called a minor-axis direction). In order to narrow the width of the beams in the major-axis direction, dynamic focus processing is performed. At the same time, in order to narrow the width of the beams in the minor-axis direction, an acoustic lens is sometimes disposed at the ultrasonic-wave emission side of an ultrasonic probe, and individual transducers are sometimes formed to have different sizes and shapes with each other for adjusting the sound-pressure distribution of the ultrasound beams (for example, refer to Patent Document 1).

However, according to the method of disposing an acoustic lens or the method of having different size and shape of transducers are used, the sound-pressure distribution of the ultrasound beams is fixed, and thus the beam width and the focal point cannot be changed at image-pickuping time. Accordingly, a plurality of ultrasonic probes having different beam widths and the focal points must be prepared, and each of the ultrasonic probes must be replaced in accordance with an imaging portion, thereby the apparatus becomes difficult to use.

An object of the present invention is to achieve an ultrasonic probe having an improved resolution of ultrasound images and ease of use, and an ultrasonic imaging apparatus.

Patent Document 1: Japanese Unexamined Patent Application Publication No. 5-41899

### DISCLOSURE OF INVENTION

According to the present invention, there is provided an ultrasonic probe including a plurality of transducers in an array for converting drive signals into ultrasonic waves to transmit the waves to an object to be inspected and converting the waves into electrical signals to receive ultrasonic waves generated from the object, wherein each of the transducers includes a plurality of oscillation elements, each of the oscillation elements has a characteristic of changing an electromechanical coupling coefficient in accordance with strength of a direct-current bias applied by being superposed on the drive signal, and an electrode of each of the transducers is connected to a terminal provided with the drive signal.

That is to say, an oscillation element having an electromechanical coupling coefficient changing in accordance with the strength of a direct-current bias can be made small compared with a piezoelectric element. Accordingly, an transducer can be formed with making intervals between the oscillation elements relatively small, and this is equivalent to subdividing the transducer, which makes it possible to improve the resolution of ultrasound images.

In particular, by making the strength of the direct-current bias applied on each oscillation element different individually, the strength of an ultrasonic wave emitted from each oscillation element differs in accordance with the strength of the direct-current bias. Accordingly, by controlling the strength of the direct-current bias applied on each oscillation element, it becomes possible to vary the strength of the ultrasound beam, or to have a desired sound-pressure distribution. As a result, it is possible to adjust the beam width of the ultrasound beam, the depth direction of a focal direction, and the position of the orientation direction in real time (for example, during an ultrasonic diagnosis) as needed, and thus an improvement in ease of use is achieved.

For example, if an transducer is formed by arranging oscillation elements in a minor-axis direction, the minor-axis direction is subdivided by the oscillation elements, and thus the resolution of an ultrasound image can be further improved. At the same time, it is possible to arbitrarily control the beam width in the minor-axis direction and the focal depth by controlling the sound-pressure distribution in the minor-axis direction.

In this case, the plurality of oscillation elements can be divided into a plurality of groups, and the electrode of each of the oscillation elements pertaining to a same group can be commonly connected. By this, it is possible to ensure the necessary strength of the ultrasonic wave for picking up an ultrasound image by determining the number of the oscillation elements pertaining to each group in consideration of the strength of the ultrasonic wave emitted from a single oscillation element.

Also, a plurality of oscillation elements may be divided into a plurality of groups in a minor-axis direction, and the electrode of each of the oscillation elements pertaining to the same group may be commonly connected. Also, a plurality of oscillation elements may be formed at equal intervals, the oscillation elements may be divided into a plurality of groups having an equal number of the oscillation elements, and the electrode of each of the oscillation elements pertaining to the same group are commonly connected. Also, a plurality of oscillation elements may be divided into a plurality of groups in a major-axis direction.

Also, a plurality of oscillation elements may be divided into a plurality of groups, the number of the oscillation elements pertaining to each of the divided groups may increase for each group as the element goes near a center of an ultrasonic aperture, and the electrode of each of the oscillation elements pertaining to the same group may be commonly connected. Also, the terminal connected to a electrode of the oscillation element may be connected to a power source through switching means.

Also, the oscillation elements may be formed by a material including a semiconductor compound. For example, the oscillation element may include a semiconductor substrate, a frame body made of a semiconductor compound placed on the semiconductor substrate, a film body made of a semiconductor compound disposed by closing the aperture of the frame body, and an electrode connected to the semiconductor substrate and the film body.

Also, according to the present invention, there is provided an ultrasonic imaging apparatus including: an ultrasonic probe described above; transmitting means for supplying drive signals to the oscillation elements of the ultrasonic probe; receiving means for processing electrical signals output from the oscillation elements; and image processing means for reconstructing an ultrasound image based on signals output from the receiving means; wherein bias means applying a direct-current bias on the oscillation elements by superposing the bias on the drive signal is connected to electrodes of the oscillation elements through the terminal.

In this case, the bias means may include a direct-current power source, distribution means for dividing a direct-current bias provided from the direct-current power source, and switching means for applying each direct-current bias supplied from the distribution means to electrodes of the oscillation elements in accordance with a control command through the terminal.

Also, a plurality of the oscillation elements may be divided into a plurality of groups, and the bias means may apply a direct-current bias having different strength for each of the groups to each of the oscillation elements. At this time, the plurality of oscillation elements are preferably divided into a plurality of groups in a minor-axis direction. Also, the plurality of oscillation elements may be divided into a plurality of groups in a major-axis direction. Also, the bias means may apply a direct-current bias increasing for each group as the element gets closer a center of an ultrasonic aperture. Also, the bias means may apply a direct-current bias to each oscillation element such that an electromechanical coupling coefficient of each of the oscillation elements increases as the element gets closer a center of a minor-axis direction. Also, a plurality of oscillation elements may be divided into a plurality of groups, and the bias means may select the oscillation element to which a direct-current bias is applied for each group in accordance with a distance from the ultrasonic probe to an imaging portion.

Also, it is possible to include storage means for storing signal strength of an ultrasonic wave transmitted from each of the oscillation elements before starting ultrasonic imaging and correction control means for generating a command to correct an electromechanical coupling coefficient of each of the oscillation elements based on the signal strength to a setting value. When ultrasonic imaging is performed, the bias means may apply a direct-current bias corrected based on the correction command to each of the oscillation elements.

Also, the bias means may alternatively apply a direct-current bias applied to each of the oscillation elements when an ultrasonic wave is transmitted from each of the oscillation elements to the object, or apply a direct-current bias to each of the oscillation elements when ultrasonic waves generated from the object are received by each of the oscillation elements.

Also, a plurality of oscillation elements may be divided into a plurality of groups, and the bias means may apply a direct-current bias having weight for each group symmetrically with respect to a center of an ultrasonic aperture in a minor-axis direction or in a major-axis direction to each of the oscillation elements. Also, a plurality of oscillation elements may be divided into a plurality of groups, and the bias means may apply a direct-current bias having weight for each group asymmetrically with respect to a center of an ultrasonic aperture in a minor-axis direction or in a major-axis direction to each of the oscillation elements.

Also, according to the present invention, there is provided a method of ultrasonic imaging including the steps of: applying a direct-current bias to a plurality of oscillation elements possessed by each transducer arrayed in an ultrasonic probe and changing an electromechanical coupling coefficient of each of the oscillation elements to a setting value; supplying a drive signal to each of the oscillation elements by superposing the drive signal on the direct-current bias and transmitting an ultrasonic wave to an object to be inspected from each of the oscillation elements; and receiving an ultrasonic wave generated by the object by each of the oscillation elements to convert the wave into an electrical signal and reconstructing an ultrasound image based on the converted electrical signal.

### BRIEF DESCRIPTION OF THE DRAWINGS

FIG. 1 is a block diagram illustrating the configuration of an ultrasonic imaging apparatus of a first embodiment to which the present invention is applied.

FIG. 2 is a perspective view of an ultrasonic probe of FIG. 1.

FIG. 3 is an enlarged perspective view of an transducer of FIG. 2.

FIG. 4 is a longitudinal sectional view of an oscillation element of FIG. 3.

FIG. 5 is a diagram illustrating the operation of the oscillation element of FIG. 4.

FIG. 6 is a diagram showing the configuration of the bias means of FIG. 1.

FIG. 7 is an explanatory diagram showing a sound-pressure distribution in a minor-axis direction of an ultrasonic beam by the ultrasonic imaging apparatus of FIG. 1.

FIG. 8 is an explanatory diagram showing a sound-pressure distribution in a minor-axis direction of an ultrasonic beam by an ultrasonic imaging apparatus of a second embodiment to which the present invention is applied.

FIG. 9 is an explanatory diagram showing a sound-pressure distribution in a minor-axis direction of an ultrasonic beam by an ultrasonic imaging apparatus of a third embodiment to which the present invention is applied.

FIG. 10 is an explanatory diagram showing a sound-pressure distribution in the major-axis direction of an ultrasonic beam by an ultrasonic imaging apparatus of a fourth embodiment to which the present invention is applied.

FIG. 11 is an explanatory diagram showing sound-pressure distributions in a minor-axis direction and in a major-axis direction of an ultrasonic beam by an ultrasonic imaging apparatus of a fifth embodiment to which the present invention is applied.

FIG. 12 is a configuration diagram showing correction control means of a sixth embodiment to which the present invention is applied.

FIG. 13 is an explanatory diagram showing the effect of the correction control means of FIG. 12.

### BEST MODE FOR CARRYING OUT THE INVENTION

### First Embodiment

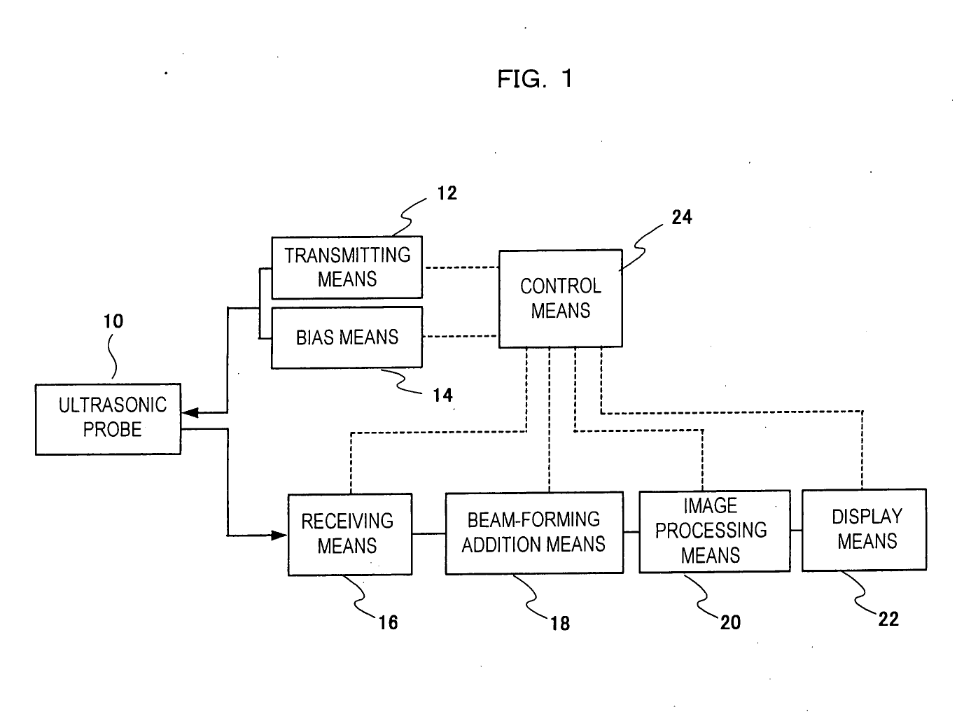

A description will be given of a first embodiment of an ultrasonic probe to which the present invention is applied and an ultrasonic imaging apparatus with reference to the drawings. FIG. 1 is a block diagram illustrating the configuration of an ultrasonic imaging apparatus of the first embodiment to which the present invention is applied.

As shown in FIG. 1, the ultrasonic imaging apparatus includes an ultrasonic probe 10 including an array of a plurality of transducers for converting drive signals into ultrasonic waves to transmit the waves to an object to be inspected and converting the waves into electrical signals to receive ultrasonic waves generated from the object, transmitting means 12 for supplying a drive signal to the ultrasonic probe 10, bias means 14 for applying a direct-current bias by superposing the bias on the drive signal supplied to the ultrasonic probe 10, receiving means 16 for processing an electrical signal (in the following, called a reflection-echo signal) output from the ultrasonic probe 10, beam-forming addition means 18 for performing digital beam-forming and addition processing on the reflection echo signal output from the receiving means 16, image processing means 20 for reconstructing an ultrasound image based on the reflection-echo signal output from the beam-forming addition means 18, display means 22 for displaying an ultrasound image output from the image processing means 20, etc. Also, the ultrasonic imaging apparatus has control means 24 for outputting a control command to the transmitting means 12, the bias means 14, the receiving means 16, the beam-forming addition means 18, the image processing means 20, and the display means 22.

In such an ultrasonic imaging apparatus, the transmitting means 12 supplies drive signals to the ultrasonic probe 10 that is in contact with an object to be inspected. Each transducer of the ultrasonic probe 10 transmits an ultrasonic wave to the object by the supplied drive signal. The ultrasonic wave generated from the object is received by each transducer of the ultrasonic probe 10. The reflection echo signal output from the ultrasonic probe 10 is subjected to receiving processing such as amplification, analog-digital conversion, by the receiving means 16. The reflection echo signal which was subjected to the receiving processing is subjected to beam-forming and addition by the beam-forming addition means 18. The reflection echo signal which was subjected to the beam-forming and addition is reconstructed into an ultrasound image (for example, a diagnosis image such as a tomogram, a blood-flow image) by the image processing means 20. The reconstructed diagnosis image is displayed to the display means 22.

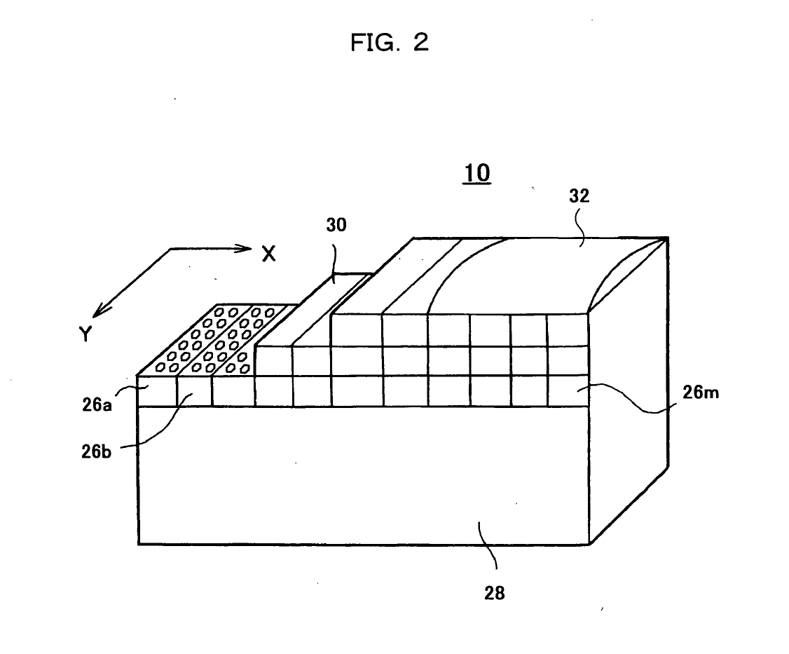

FIG. 2 is a perspective view of the ultrasonic probe 10 of FIG. 1. As shown in FIG. 2, the ultrasonic probe 10 is formed in a one-dimensional array in which a plurality of transducers 26a to 26m (m: a natural number of 2 or more) are disposed in a strip-like form. However, the present invention can be applied to an ultrasonic probe having another form such as a two-dimensional array type including a two-dimensional array of transducers, a convex type including transducers in a fan-like form. A matching layer 30 is disposed by being laminated to the ultrasonic-wave emission side of transducers 26a to 26m. An acoustic lens 32 is disposed on the side of an object to be inspected of the matching layer 30. In this regard, a form without disposing the acoustic lens 32 is allowed. Also, a backing material 28 is disposed by being overlapped on the back surface side of the transducers 26a to 26m.

The transducers 26a to 26m convert drive signals supplied from the transmitting means 12 into ultrasonic waves to transmit the ultrasonic waves to an object to be inspected, and receives the ultrasonic waves generated from the object to convert the waves into electrical signals. The backing material 28 restrains excessive oscillations of the transducers 26a to 26m by absorbing the propagation of the ultrasonic waves emitted at the back surface side of the transducers 26a to 26m. The matching layer 30 performs the matching of acoustic impedance between the transducers 26a to 26m and the object, thereby improving the transmission efficiency of the ultrasonic waves. The acoustic lens 32 is formed by being curved toward the object side, and makes the ultrasound beams emitted from the transducers 26a to 26m converge. In this regard, the arranging direction of the transducers 26a to 26m is called the major-axis direction X, and the direction orthogonal to the major-axis direction X is called as the minor-axis direction Y.

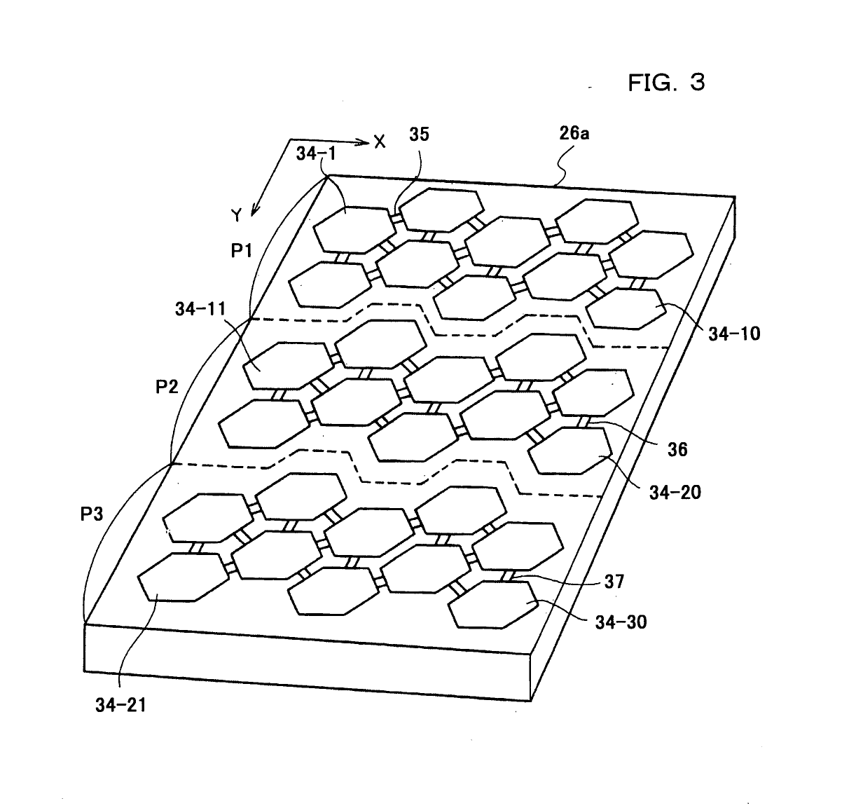

FIG. 3 is an enlarged perspective view of the transducer 26a of FIG. 2. As shown in FIG. 3, the transducer 26a is formed with a plurality of oscillation elements 34-1 to 34-30. The oscillation elements 34-1 to 34-30 are electro-acoustic transformation elements having electromechanical coupling coefficients, that is to say, transmitting and receiving sensitivities, which change by the strength of the applied direct-current biases.

The oscillation elements 34-1 to 34-30 are formed by being disposed at equal intervals in the major-axis direction X and in the minor-axis direction Y. However, the elements may be formed at irregular intervals. Also, the oscillation elements 34-1 to 34-30 are divided into three groups (in the following, called sections) P1 to P3 in the minor-axis direction Y. The oscillation elements 34-1 to 34-10 pertaining to the section P1 are commonly connected to an electrode 35. The oscillation elements 34-11 to 34-20 pertaining to the section P2 are commonly connected to an electrode 36. The oscillation elements 34-21 to 34-30 pertaining to the section P3 are commonly connected to an electrode 37.

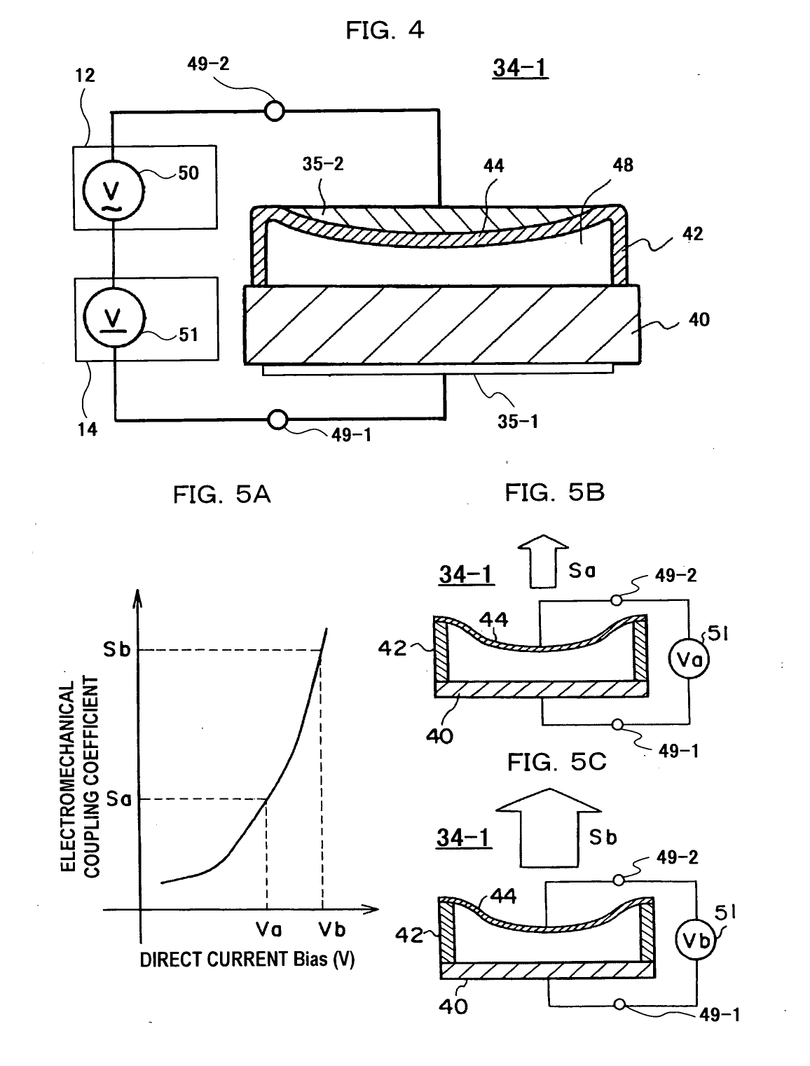

FIG. 4 is a longitudinal sectional view of the oscillation element 34-1 of FIG. 3. As shown in FIG. 4, the oscillation element 34-1 is formed by a substrate 40, a frame body 42 formed on the surface of the object side of the substrate 40, a film body 44 disposed by closing the aperture of the frame body 42, etc. The substrate 40, the frame body 42, and the film body 44 are formed by including a semiconductor compound (for example, a silicon compound). An internal space 48 is partitioned by the frame body 42 and the film body 44. The internal space 48 is kept in a state having a predetermined degree of vacuum or a state of being filled up with a predetermined gas. Also, the oscillation element 34-1 has an electrode 35-1 disposed on the surface of the back face side of the substrate 40 and an electrode 35-2 disposed on the surface of the object side of the film body 44. The electrode 35-1 is connected to a drive-signal power source 50 of the transmitting means 12 through a connection terminal 49-1. The electrode 35-2 is connected to a direct-current bias power source 51 of the bias means 14 through a connection terminal 49-2.

The oscillation element 34-1 is produced by micro fabrication by a semiconductor process. For example, a silicon wafer to be a substrate 40 is provided. An oxide film is formed on the silicon wafer in a wet atmosphere. The substrate on which the oxide film has been formed is subjected to pattern forming, resist application, etc., and then is subjected to etching processing to form the frame body 42. Predetermined gas is filled in the inside of the formed frame body 42. Nickel (Ni) is deposited on the frame body 42 by LPCVD (Low Pressure Chemical Vapor Deposition), thereby forming the film body 44. The electrodes 35-1 and 35-2 are formed by depositing metal electrode. A plurality of oscillation elements are formed on the silicon wafer by those processes. Each of the formed oscillation elements has a diameter of a few micrometers (for example, 10 μm). The wafer on which the oscillation elements are formed is cut into a plurality of pieces as the transducers 26a to 26m by MEMS (Micro Electro Mechanical System). The transducers 26a to 26m that have been cut are arranged on the backing material 28, and then are bonded on a probe-head substrate. The drive-signal power source 50 and the direct-current bias power source 51 are connected to the probe-head substrate through the connection terminals 49-1 and 49-2. In this regard, the matching layer 30, the acoustic lens 32, etc., are also attached to the transducers 26a to 26m.

To such oscillation elements 34-1 to 34-30, for example, CMUT (Capative Micromachined Ultrasonic Transducer: IEEE Trans. UItrason. Ferroelect. Freq. Contr. Vol15 pp. 678-690 May 1998) can be applied.

FIG. 5 is a diagram illustrating the operation of the oscillation element 34-1 of FIG. 4. For example, a direct-current bias voltage Va is applied to the oscillation element 34-1 by the direct-current bias power source 51. An electric field is generated in the internal space 48 of the oscillation element 34-1 by the applied bias voltage Va. The generated electric field increases the tension of the film body 44, and thus the electromechanical coupling coefficient of the oscillation element 34-1 becomes Sa (FIG. 5A, FIG. 5B). When a drive signal is supplied to the oscillation element 34-1 from the drive-signal power source 50, the supplied drive signal is converted into an ultrasonic wave based on the electromechanical coupling coefficient Sa. Also, when the oscillation element 34-1 receives the ultrasonic waves generated from the object, the film body 44 of the oscillation element 34-1 is excited based on the electromechanical coupling coefficient Sa. The excitation of the film body 44 causes the capacity of the internal space 48 to change. The changed capacity is captured as an electrical signal.

On the other hand, when a bias voltage Vb (Vb>Va) is applied to the oscillation element 34-1 instead of the bias voltage Va, the tension of the film body 44 is changed by the applied bias voltage Vb. Thus, the electromechanical coupling coefficient of the oscillation element 34-1 becomes Sb (Sb>Sa) (FIG. 5A, FIG. 5C). When a drive signal is supplied to the oscillation element 34-1 from the drive-signal power source 50, the supplied drive signal is converted into an ultrasonic wave based on the electromechanical coupling coefficient Sb.

As above, it is possible to change the degree of the tension of the film body 44 by controlling the bias voltage value applied to the oscillation element 34-1. The degree of the tension of the film body 44 causes the electromechanical coupling coefficient to change. Accordingly, it is possible to adjust the strength (for example, the magnitude of amplitude) of the ultrasonic wave transmitted and received by the oscillation element 34-1 by changing the electromechanical coupling coefficient by controlling the bias voltage value. As a result, it becomes possible to arbitrarily change the sound-pressure distribution of the ultrasound beams by adjusting the strength of each of the ultrasonic waves transmitted from and received to a plurality of the oscillation elements 34-1 to 34-30.

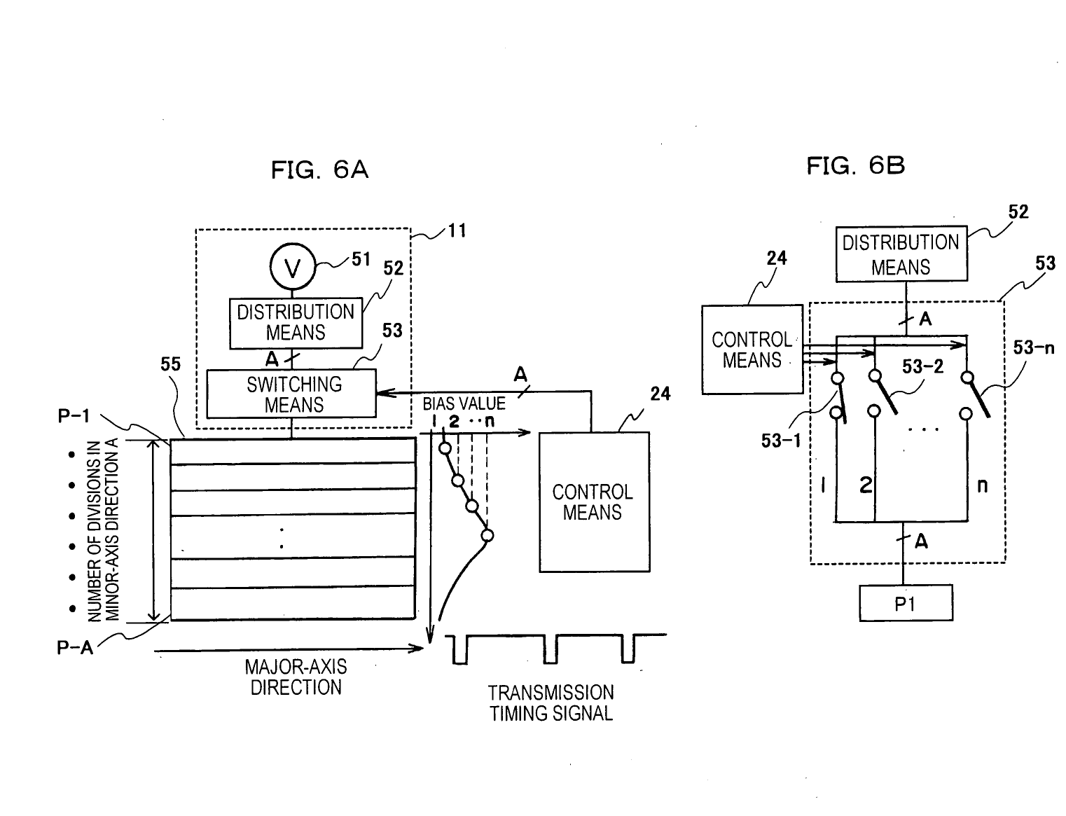

FIG. 6 is a diagram showing the configuration of the bias means 14 of FIG. 1. As shown in FIG. 6A, the bias means 14 includes the direct-current bias power source 51, distribution means 52 for dividing the direct-current bias given from the direct-current bias power source 51, and switching means 53 for applying each direct-current bias supplied from the distribution means 52 to the electrodes 35 to 37 of the oscillation elements 34-1 to 34-30 in accordance with a control command of the control means 24 through connection terminals (for example, connection terminals 35-1 and 35-2). As shown in FIG. 6B, the switching means 53 has a plurality of switches 53-1 to 53-n connecting to the transducer 55.

For convenience of explanation, FIG. 6 shows an example in which the transducer 55 is divided into A pieces of sections P1 to PA (A: a natural number of 2 or more) in the minor-axis direction Y. In this regard, a plurality of oscillation elements are formed in each of the sections P1 to PA. First, when the direct-current bias power source 51 generates a direct-current bias, the generated direct-current bias is divided by the distribution means 52. Each of the divided direct-current bias is supplied to the switching means 53. At the same time, by inputting a transmission timing signal of the ultrasonic wave into the control means 24, a control command is generated based on the input transmission timing signal. The generated control command is output to the switching means 53. A predetermined switch (for example, the switch 53-1) is turned on based on the output control command. Accordingly, the direct-current bias supplied to the switching means 53 is independently applied to an electrode of a section (for example, the section P1) of the transducer 55 through a predetermined switch (for example, the switch 53-1).

The switching means 53 is provided corresponding to the number of the sections P1 to PA. Accordingly, the value of the direct-current bias applied to the electrode of each of the sections P1 to PA is adjusted by the number of closings of the switches 53-1 to 53-n of each switching means 53. For example, for the section P1 located at the end of the transducer 55 in the minor-axis direction Y, a bias voltage Va is applied by turning only the switch 53-1 on. For the section P (A/2) located at the center of the transducer 55 in the minor-axis direction Y, a bias voltage (Va×n) is applied to the electrode by turning all the switches 53-1 to 72-n on. In this manner, by changing the number of switches 53-1 to 72-n to be turned on in each switching means 53, it is possible to make the bias voltage to be applied to each section of the transducer 55 different for each section.

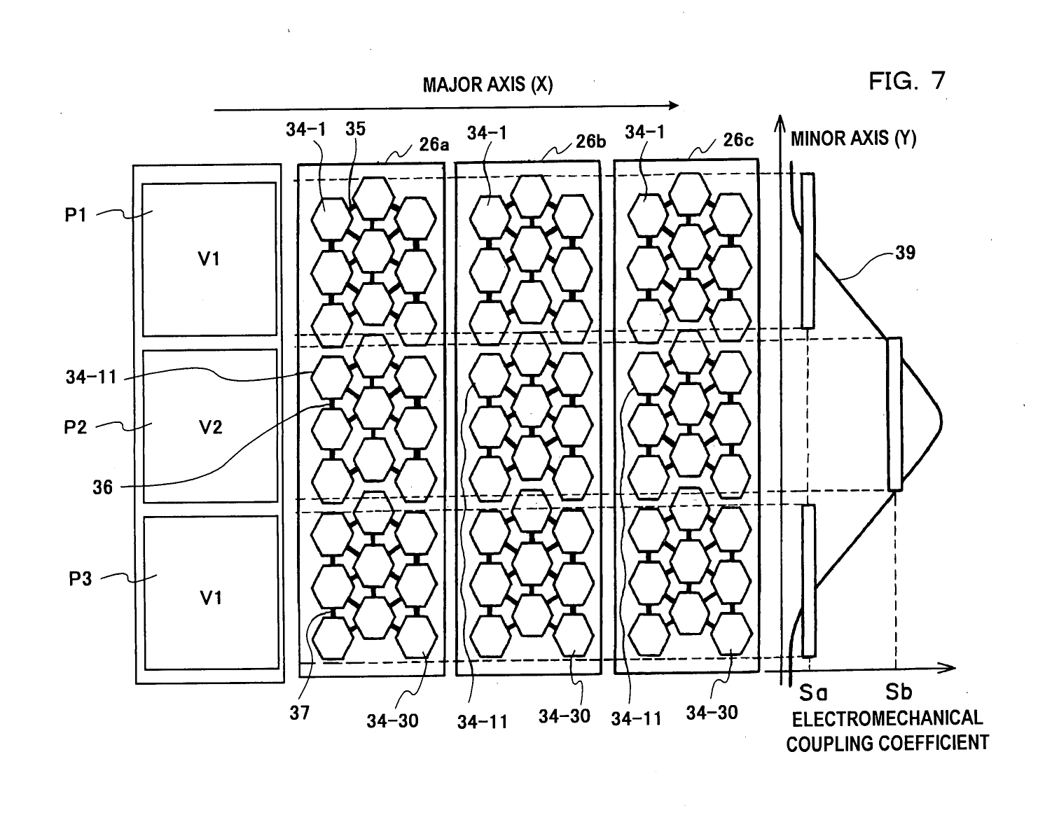

FIG. 7 is an explanatory diagram showing a sound-pressure distribution in a minor-axis direction of an ultrasonic beam by the ultrasonic imaging apparatus of FIG. 1. In this regard, for convenience of explanation, a description will be given of an example of three transducers 26a to 26c. However, the number of transducers can be increased appropriately. As shown in FIG. 7, the transducers 26a to 26c are arranged in a line in the major-axis direction X. The transducer 26a is formed with a plurality of oscillation elements 34-1 to 34-30. The plurality of oscillation elements 34-1 to 34-30 are divided into three sections P1 to P3 in the minor-axis direction Y. The oscillation elements 34-1 to 34-10 pertaining to the same section (for example, the section P1) are commonly connected to the electrode 35. This arrangement is the same for the transducers 26b and 26c.

When a bias voltage V1 is applied to the electrode 35 of the section P1 and the electrode 37 of the section P3, the electromechanical coupling coefficients of the oscillation elements 34-1 to 34-10 and 34-21 to 34-30 pertaining to the sections P1 and P3, respectively, become Sa. At the same time, when a bias voltage V2 (V2>V1) is applied to the electrode 36 of the section P2, the electromechanical coupling coefficients of the oscillation elements 34-11 to 34-20 pertaining to the sections P2 become Sb (Sa>Sb).

That is to say, when the bias voltage value is increased for each section as the position gets closer the center of the ultrasonic aperture, as shown in FIG. 7, the electromechanical coupling coefficient of the transducer increases for each section as the position gets closer the center in the minor-axis direction Y. Each of the transducers 26a to 26c emits an ultrasonic wave based on such an electromechanical coupling coefficient. By this means, even when common drive signals (for example, drive signals having an equal amplitude) are input into each of the oscillation elements 34-1 to 34-30, the sound-pressure distribution of the ultrasound beams is represented as a weighting function 39 having an increasing value as the position gets closer the center in the minor-axis direction Y as shown by the diagram in FIG. 7. In summary, a direct-current bias applied to each of the sections P1 to P3 is made different for each section, thus the value of the electromechanical coupling coefficient of each of the transducers 26a to 26c is weighted for each section in the minor-axis direction, and thereby the sound-pressure distribution of the ultrasound beams is controlled.

As described above, according to the present embodiment, the oscillation elements 34-1 to 34-30 having the electromechanical coupling coefficient values changing in accordance with the direct-current bias value are formed to have, for example, a few micrometers in size. Thus, the oscillation element becomes finer than piezoelectric elements made of a piezoelectric material. Accordingly, by forming each transducer (for example, transducer 26a) with the intervals of the oscillation elements 34-1 to 34-30 made relatively small, it becomes equivalent to the fractionization of the transducer. Thus, it is possible to improve the resolution of an ultrasound image.

In particular, by making the value of the direct-current bias applied on each of the oscillation elements 34-1 to 34-30 different for section or for each oscillation element, the strength of an ultrasonic wave emitted from each of the oscillation elements 34-1 to 34-30 becomes different in accordance with the value of the direct-current bias. Accordingly, by controlling the strength of the direct-current bias applied on each oscillation element, it becomes possible to vary the strength of the ultrasound beam, or to have a desired sound-pressure distribution. As a result, it is possible to adjust the beam width of an ultrasound beam, the depth direction of a focal direction, and the position of the orientation direction in real time (for example, during an ultrasonic diagnosis) as needed, and thus ease of use is improved.

For example, as shown in FIG. 3, if the transducer 26a is formed by arranging the oscillation elements 34-1 to 34-30 in the minor-axis direction Y, it becomes equivalent that the minor-axis direction Y is subdivided by the oscillation elements 34-1 to 34-30, and thus the resolution of an ultrasound image can be further improved. Furthermore, it is possible to arbitrarily control the beam width in the minor-axis direction Y and the focal depth by controlling the sound-pressure distribution.

Also, as shown in FIG. 3 and FIG. 7, the oscillation elements 34-1 to 34-30 are divided into a plurality of the sections P1 to P3, and the electrode (for example, the electrode 35) of each of the oscillation elements (for example, the oscillation elements 34-1 to 34-10) pertaining to the same section (for example, the section P1) are commonly connected. By this, it is possible to ensure the necessary strength of the ultrasonic wave for picking up an ultrasound image by increasing the number of the oscillation elements pertaining to each section even when the strength of the ultrasonic wave emitted from a single oscillation element (for example, the oscillation element 34-1) is very weak.

Also, when the strength of the ultrasonic wave emitted from a single oscillation element (for example, the oscillation element 34-1) is strong, bias voltages having a different value for each of the oscillation elements 34-1 to 34-30 in place of for each section may be applied. By this, the adjustment range of the sound-pressure distribution of the ultrasound beams can be still further subdivided. Also, since the transducers 26a to 26c are divided into a plurality of sections P1 to P3 in the minor-axis direction Y, it is possible to adjust the sound-pressure distribution of the ultrasound beams in the minor-axis direction Y for each section.

The present invention has been described based on the first embodiment. However, the present invention is not limited to this. For example, the transducers in FIG. 3 and FIG. 7 have the same number of oscillation elements pertaining to the same section. However, the number of the transducers may increase as the position gets closer the center of the ultrasonic aperture. By this means, it is possible to reduce the effect of the end part of the ultrasonic aperture, and thus it is possible to increase the S/N of an ultrasound image.

Also, the beam width in the major-axis direction X and the focal depth of the transducers 26a to 26c shown in FIG. 7 can be adjusted by performing dynamic focus by the beam forming addition means 18 on the reflection echo signal output from each of the transducers 26a to 26c. In this case, the oscillation elements 34-1 to 34-30 may be formed by being arranged in the major-axis direction X of each transducer (for example, the transducer 26a) along with the dynamic focusing technique or in place of the technique, and the beam width in the major-axis direction X and the focal depth of the ultrasound beams may be controlled by applying direct-current biases having different strength to each oscillation element. Also, the oscillation elements 34-1 to 34-30 may be divided into a plurality of groups (sections) in the major-axis direction X, direct-current biases having a different value for each group may be applied to each of the oscillation elements 34-1 to 34-30, and thus the sound-pressure distribution of the ultrasound beams in the major-axis direction X is controlled for each section.

Also, according to the present embodiment, by making the direct-current bias applied to each of the oscillation elements 34-1 to 34-30 different, if the transmitting means 12 supplies a common drive signal (for example, a drive signal having the same amplitude) to the ultrasonic probe 10, it is possible to control the sound-pressure distribution of the ultrasound beams. Accordingly, the circuit of the transmitting means 12 comes to have a simpler configuration than a transmitting system circuit generating drive signals with individually different amplitudes.

Also, as shown in FIG. 3, each of the oscillation elements 34-1 to 34-30 is configured to be a hexagonal thin plate in shape. By configuring the element to be a hexagon in this manner, it is possible to narrow the clearance (gap) among the oscillation elements 34-1 to 34-30. Accordingly, it is possible to closely dispose the oscillation elements 34-1 to 34-30 in an array. As a result, the number of arrays per unit area of the oscillation elements 34-1 to 34-30 becomes large, and thus a desired strength of the ultrasound beams is ensured. Also, when the surface shape of the transducer 26a is a curved surface, by bending the electrodes 35 to 37 corresponding to the curved surface, it is possible to arrange the oscillation elements 34-1 to 34-30 having flat surfaces in the transducer 26a. However, each of the oscillation elements 34-1 to 34-30 is not limited to be a hexagon-like form, and may be a polygon such as an octagon, and a circle-like form. Also, each of the oscillation elements 34-1 to 34-30 is formed to have a diameter of 10 μm, for example. By forming only the oscillation elements arranged on the surface end part of the transducer 26a, it is possible to further increase the density of the oscillation elements 34-1 to 34-30. Also, in FIG. 2, a description has been given of an example in which a rectangular ultrasonic aperture is formed by a plurality of transducers 26a to 26m. However, the present invention can be applied to the case in which a circular ultrasonic aperture is formed by arranging disc-shaped transducers.

Also, for switching means shown in FIG. 6, it is possible to adjust the value of the bias voltage finely by increasing the number of the switches 53-1 to 53-n. Also, the number of control wiring lines transmitting a command output from the control means 24 corresponds to the number of sections A of the transducer 55. However, it is not always necessary to make both of the numbers match. For example, when the ultrasound beams are formed symmetrically about the middle position of the ultrasound beams in the minor-axis direction, it is possible to make the number of control wiring lines half of the number of sections A.

### Second Embodiment

A description will be given of a second embodiment of an ultrasonic probe to which the present invention is applied and an ultrasonic imaging apparatus with reference to the drawings. The present embodiment is different from the first embodiment in the point that a plurality of groups (sections) of each transducer is further divided into a plurality of groups, and a different direct-current bias value is applied to each group. Accordingly, the description of the same portion as that of the first embodiment is omitted, and a description will be given on the different points. In this regard, a description will be given by adding the same letters and numerals to the mutually corresponding portions.

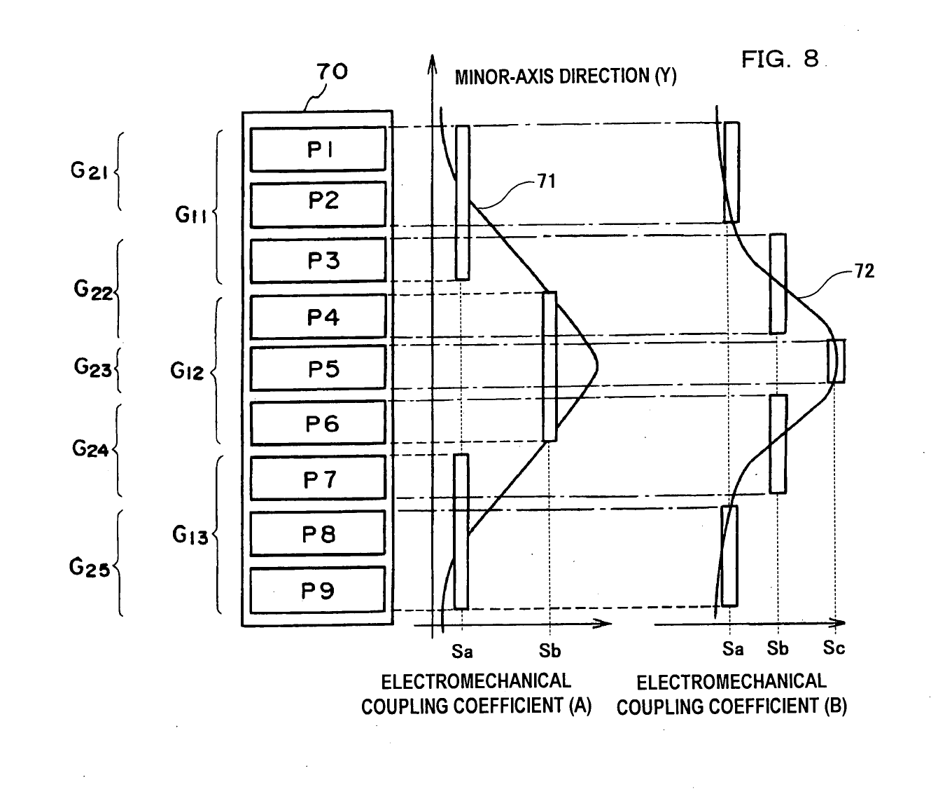

FIG. 8 is an explanatory diagram showing a sound-pressure distribution in a minor-axis direction of an ultrasonic beam by an ultrasonic imaging apparatus of the second embodiment to which the present invention is applied. As shown in FIG. 8, an transducer 70 is formed with a plurality of oscillation elements. The plurality of the oscillation elements are divided into a plurality of sections P1 to P9 in the minor-axis direction Y. In this regard, each of the oscillation elements is formed in the same form as that shown in FIG. 4. The plurality of sections P1 to P9 are divided into three groups G11, G12, and G13 in the minor-axis direction Y. For example, the group G11 is formed by three sections P1 to P3.

By applying a bias voltage Va to the sections P1 to P3 pertaining the group G11 and the sections P7 to P9 pertaining the group G13, the electromechanical coupling coefficients of the oscillation elements pertaining to the sections P1 to P3 and P7 to P9 become Sa. At the same time, by applying a bias voltage Vb to the sections P4 to P6 pertaining the group G12, the electromechanical coupling coefficients of the oscillation elements pertaining to the sections P4 to P6 become Sb. That is to say, as shown in FIG. 8A, the electromechanical coupling coefficient of the transducer increases in the minor-axis direction Y for each group as the position gets closer the central part in the minor-axis direction Y. The ultrasonic waves are emitted from the transducer 70 based on these electromechanical coupling coefficients. Thus, even when a common drive signal is input into each of the oscillation elements, the sound-pressure distribution of the ultrasound beams is represented as a weighting function 71 which increases its value as the position gets closer the central part in the minor-axis direction Y as shown in FIG. 8.

Also, as shown in FIG. 8, transducer 70 may be divided into five sections, that is, a group G21 including sections P1 and P2, a group G22 including sections P3 and P4, a group G23 including a section P5, a group G24 including sections P6 and P7, and a group G25 including sections P8 and P9.

By applying a bias voltage Va to the sections P1 and P2 pertaining the group G21 and the sections P8 and P9 pertaining the group G25, the electromechanical coupling coefficients of the oscillation elements pertaining to the sections P1, P2, P8, and P9 become Sa. By applying a bias voltage Vb to the sections P3 and P4 pertaining the group G22 and the sections P6 and P7 pertaining the group G24, the electromechanical coupling coefficients of the oscillation elements pertaining to the sections P3, P4, P6, and P7 become Sb. By applying a bias voltage Vc (Vc>Vb>Va) to the section P5 pertaining the group G23, the electromechanical coupling coefficients of the oscillation elements pertaining to the section P5 become Sc. That is to say, as shown in FIG. 8B, the electromechanical coupling coefficients of the transducer increase in the minor-axis direction Y for each group as the position gets closer the central part in the minor-axis direction Y. By emitting the ultrasonic waves from the transducer 70 based on these electromechanical coupling coefficients, even when a common drive signal is input into each of the oscillation elements, the sound-pressure distribution of the ultrasound beams can be represented as a weighting function 72 which increases its value as the position gets closer the central part in the minor-axis direction Y.

According to the present embodiment, as is understood from the weighting functions 71 and 72 shown in FIG. 8, by changing the number of sections constituting a group, it becomes possible to minutely control the sound-pressure distribution of the ultrasound beams. That is to say, by appropriately increasing and decreasing the number of the sections constituting a group, it is possible to subdivide the adjustment range of the sound-pressure distribution of the ultrasound beams. In this regard, the way of dividing a group may be appropriately determined in consideration of the strength of the ultrasonic waves transmitted for each section. Also, a description has been given of an example in which the sections of the transducer 70 is divided into groups. However, the value of the bias voltage Vc applied to each oscillation element may be controlled in place of the division into groups, and the electromechanical coupling coefficients of the transducer may increase as the position gets closer the central part in the minor-axis direction Y. In this regard, the present embodiment can be appropriately combined with the first embodiment and the variations thereof.

### Third Embodiment

A description will be given of a third embodiment of an ultrasonic probe to which the present invention is applied and an ultrasonic imaging apparatus with reference to the drawings. The present embodiment is different from the first to the second embodiments in the point that a direct-current-bias applied section is changed in accordance with a focal depth. Accordingly, the description of the same portion as that of the first and the second embodiments is omitted, and a description will be given on the different points. In this regard, a description will be given by adding the same letters and numerals to the mutually corresponding portions.

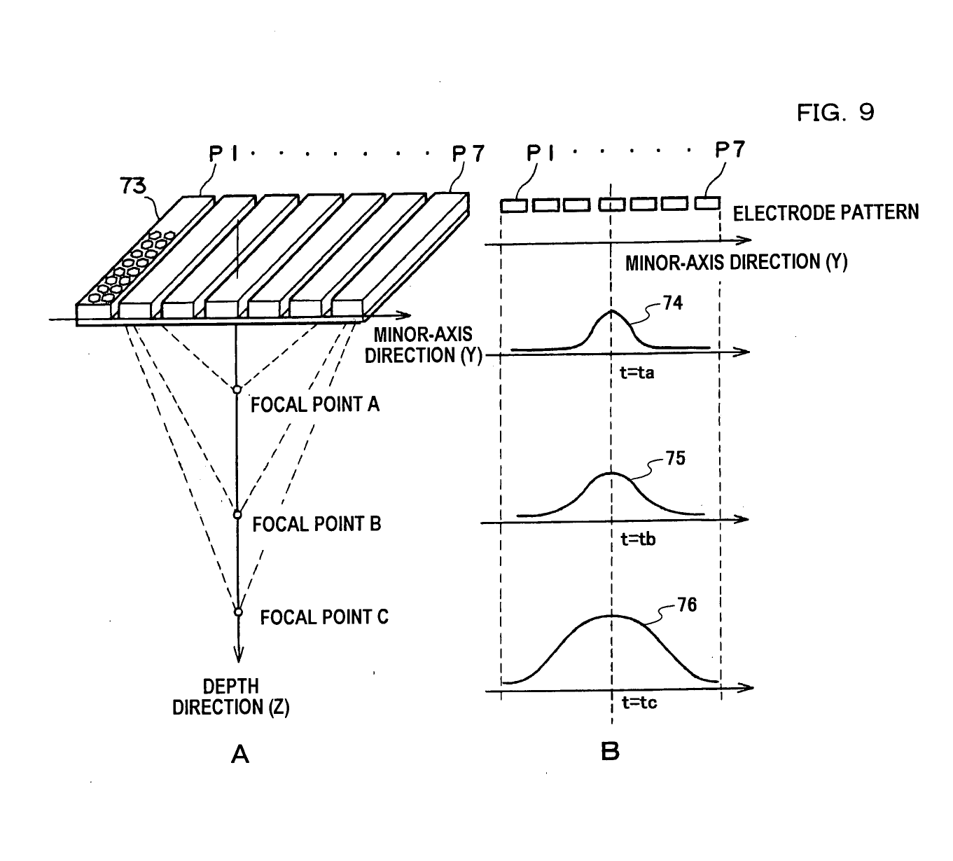

FIG. 9 is an explanatory diagram showing a sound-pressure distribution in a minor-axis direction of an ultrasonic beam by an ultrasonic imaging apparatus of a third embodiment to which the present invention is applied. As shown in FIG. 9, an transducer 73 formed by a plurality of oscillation elements is divided into 7 sections P1 to P7 in the minor-axis direction Y. Also, as focal positions of the ultrasound beams, three focal points A to C are set in the depth direction Z. In this regard, the time at which ultrasonic waves are transmitted is set to t=0. The time at which reflection echo signals generated from the focal points A, B and C are received is set to be t=ta, t=tb, and t=tc, respectively.

As shown in FIG. 9B, when a reflection echo signal generated from a focal point A is received (t=ta), the sections P3 to P5 are selected by the bias means 14 in accordance with a command of the control means 24. Predetermined values of the bias voltage are applied to the selected sections P3 to P5, respectively. Also, when a reflection echo signal generated from a focal point B is received (t=tb), the sections P2 to P6 are selected by the bias means 14 in accordance with a command of the control means 24. Predetermined values of the bias voltage are applied to the selected sections P2 to P6, respectively. Furthermore, when a reflection echo signal generated from a focal point C is received (t=tc), the sections P1 to P7 are selected. Predetermined values of the bias voltage are applied to the selected sections P1 to P7, respectively. In this regard, in the sections to which a bias voltage is not applied, the electromechanical coupling coefficients of the oscillation elements pertaining to the sections are so small that there is no impact on the beam pattern of the ultrasound beams.

According to the present embodiment, by changing the section to which a bias voltage is applied for each time when reflection echo signals generated from the focal points A to C are received, it is possible to change the ultrasonic aperture for receiving the reflection echo signals in accordance with the depth of the focal points A to C. Accordingly, it becomes equivalent to the case where a variable-aperture technique, in which the receiving aperture is automatically made smaller as the focal depth becomes shallower, is applied. Thus, it is possible to improve the direction resolution of the portion near the ultrasonic probe 10 in the minor-axis direction.

Also, as is understood from the weighting functions 74, 75, and 76 shown in FIG. 9B, by appropriately control the value of the bias voltage applied to the selected section in accordance with the focal depth, it is possible to change the strength of the ultrasound beam in accordance with the focal depth. Alternatively, it is possible to have a desired sound-pressure distribution in the minor-axis direction Y. As a result, it is possible to adjust the beam width of an ultrasound beam, the depth direction of a focal direction, and the position of the orientation direction in real time as needed, and thus ease of use is improved. In summary, by selecting the oscillation element to which a direct-current bias is applied for each section in accordance with the distance from the ultrasonic probe 10 to the imaging portion, it is possible to form the optimum ultrasound beams depending on the distance.

Also, a description has been given mainly of the operation when reflection echo signals generated from the focal points A to C are received. However, the present embodiment can be applied to the case where ultrasonic waves are transmitted from the transducer 73. For example, a section of the transducer 73 is selected in accordance with the depth of the focal position of the ultrasound beam. When a drive signal is input into the transducer 73, a bias voltage is applied to the selected section ultrasonic waves are emitted from the sections to which the bias voltage has been applied. By this means, by controlling the number of sections to be selected and by controlling the value of voltage bias, it is possible to optimize the beam shape of the ultrasound beams in accordance with the depth of the focal point.

Also, the present embodiment can be appropriately combined with the first and the second embodiments and the variations thereof.

### Fourth Embodiment

A description will be given of a fourth embodiment of an ultrasonic probe to which the present invention is applied and an ultrasonic imaging apparatus with reference to the drawings. The present embodiment is different from the first to the third embodiments in the point that a bias voltage having a different value is applied to each of the transducers arranged in the major-axis direction X in order to control the sound-pressure distribution of the ultrasound beams in the major-axis direction X. Accordingly, the description of the same portion as that of the first to the third embodiments is omitted, and a description will be given on the different points. In this regard, a description will be given by adding the same letters and numerals to the mutually corresponding portions.

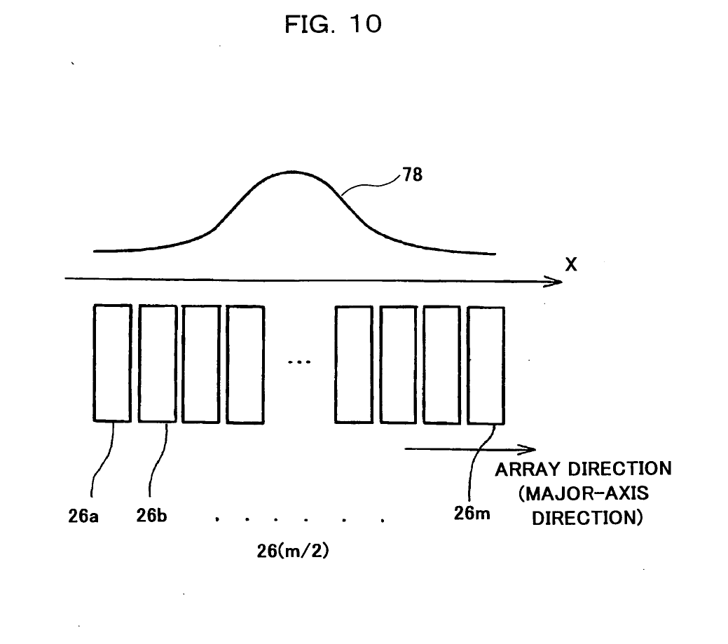

FIG. 10 is an explanatory diagram showing a sound-pressure distribution in the major-axis direction of an ultrasonic beam by an ultrasonic imaging apparatus of a fourth embodiment to which the present invention is applied. As shown in FIG. 10, transducers 26a to 26m formed by a plurality of oscillation elements are arranged in the major-axis direction X. Each of the transducers 26a to 26m is the same as that shown in FIG. 4.

In the present embodiment, a relatively large bias voltage is applied to the transducer located at the central part in the major-axis direction X. Also, a bias voltage having a smaller value for each transducer as the position goes from the central part to an end part in the major-axis direction X is applied to each transducer. For example, a relatively large bias voltage is applied to the transducer 26 (m/2). A relatively small bias voltage is applied to the transducers 26a and 26m. Thus, the sound-pressure distribution of the ultrasound beams in the major-axis direction X has a smaller strength as the position gets from the central part to an end part in the major-axis direction X as shown by the weighting function 78 in FIG. 10.

According to the present embodiment, by controlling the value of the bias voltage applied to each of the transducers 26a to 26m arranged in the major-axis direction X, it is possible to change the sound-pressure distribution of the ultrasound beams in the major-axis direction X in real time. In this regard, when controlling the sound-pressure distribution of the ultrasound beams in the major-axis direction X, a dynamic focusing technique may be used at the same time.

Also, the present embodiment can be appropriately combined with the first to the third embodiments and the variations thereof.

### Fifth Embodiment

A description will be given of a fifth embodiment of an ultrasonic probe to which the present invention is applied and an ultrasonic imaging apparatus with reference to the drawings. The present embodiment is different from the first to the fourth embodiments in the point that both of the sound-pressure distributions of the ultrasound beams in the major-axis direction X and in the minor-axis direction Y are controlled. Accordingly, the description of the same portion as that of the first to the fourth embodiments is omitted, and a description will be given on the different points. In this regard, a description will be given by adding the same letters and numerals to the mutually corresponding portions.

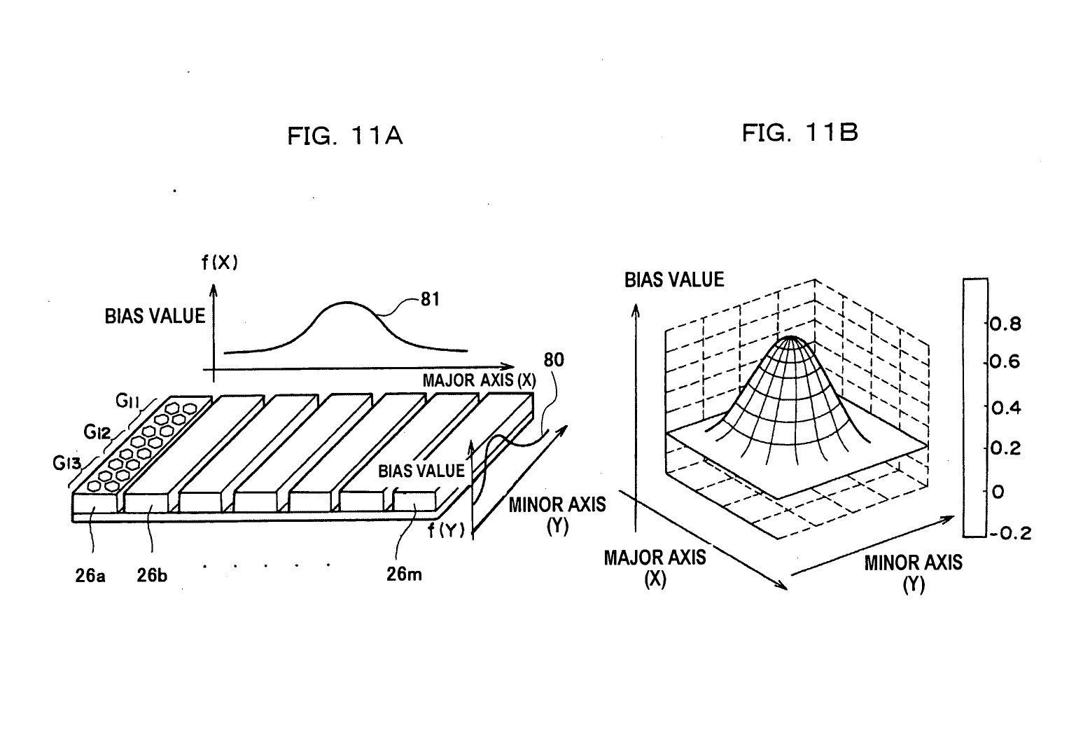

FIG. 11 is an explanatory diagram showing sound-pressure distributions in the minor-axis direction and in the major-axis direction of an ultrasonic beam by an ultrasonic imaging apparatus of a fifth embodiment to which the present invention is applied. As shown in FIG. 11A, a plurality of transducers 26a to 26m are arranged in a line. Each transducer (for example, the transducer 26a) has a plurality of oscillation elements. The oscillation elements of each transducer (for example, the transducer 26a) are divided into three sections G11, G12, and G13 in the minor-axis direction Y. In this regard, each oscillation element is the same as that shown in FIG. 4.

In the present embodiment, in the minor-axis direction Y, a bias voltage applied to the sections G11 and G13 are made relatively small, and a bias voltage applied to the section G12 is made relatively large. Thus, the sound-pressure distribution of the ultrasound beams in the minor-axis direction Y becomes the distribution represented as the weighting function 80 shown in FIG. 11A. At the same time, in the major-axis direction X, a bias voltage applied to the transducer 26 (m/2) located at the central part is made relatively large, and a bias voltage is made relatively smaller for each transducer as the position gets to an end part. Thus, the sound-pressure distribution of the ultrasound beams in the major-axis direction X becomes the distribution represented as the weighting function 81 shown in FIG. 11A.

According to the present embodiment, as shown in FIG. 11B, the values of the bias voltage applied to the transducers 26a to 26m are made to have distributions in the major-axis direction X and in the minor-axis direction Y, and thus the sound-pressure distribution of the ultrasound beams can be controlled in three dimensions. Accordingly, it becomes easy to achieve the optimum sound-pressure distribution.

Also, the present embodiment can be appropriately combined with the first to the fourth embodiments and the variations thereof.

### Sixth Embodiment

A description will be given of a sixth embodiment of an ultrasonic probe to which the present invention is applied and an ultrasonic imaging apparatus with reference to the drawings. The present embodiment is different from the first to the fifth embodiments in the point that the variations of the electromechanical coupling coefficients due to the manufacturing process of oscillation elements is corrected. Accordingly, the description of the same portion as that of the first to the fifth embodiments is omitted, and a description will be given on the different points. In this regard, a description will be given by adding the same letters and numerals to the mutually corresponding portions.

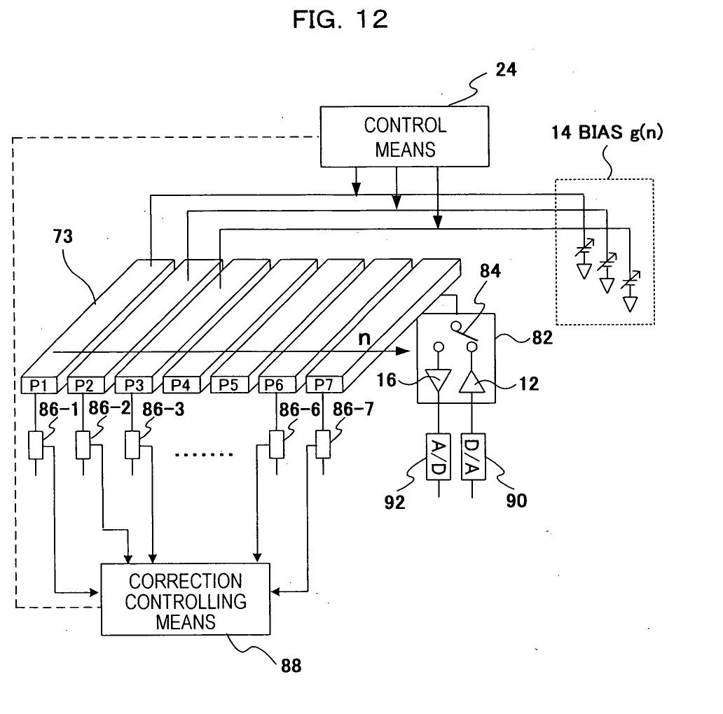

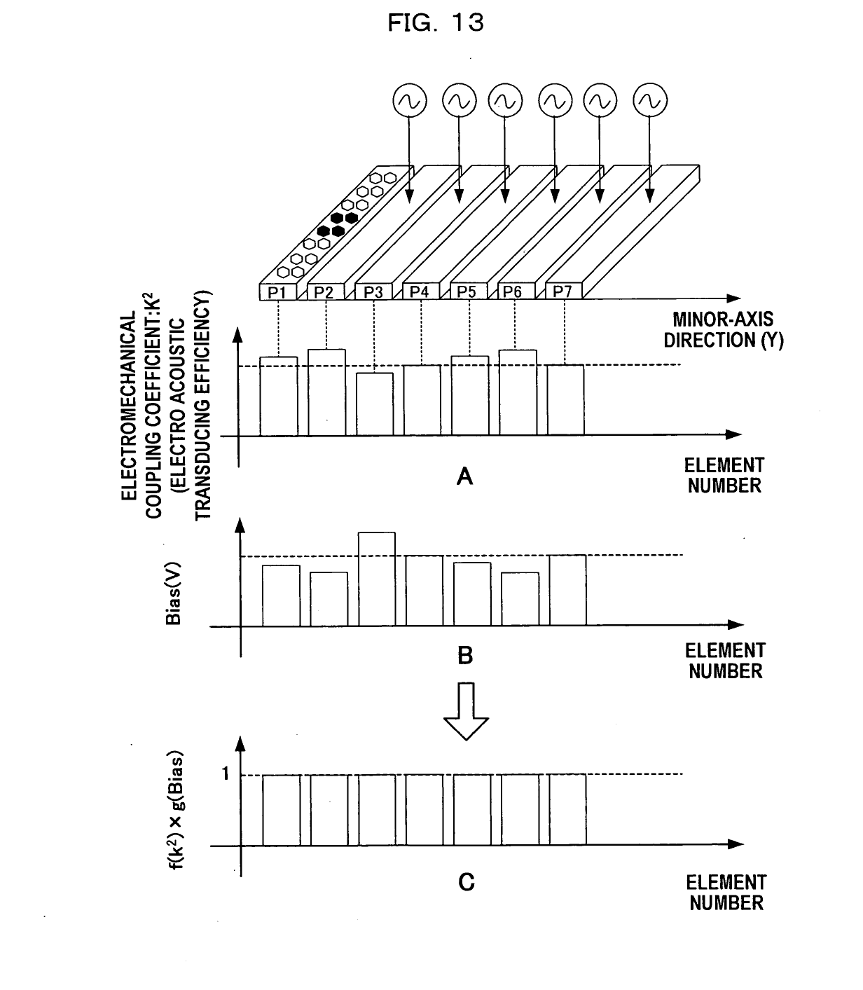

FIG. 12 is a configuration diagram showing correction control means of the present embodiment. FIG. 13 is an explanatory diagram showing the effect of the present embodiment. In this regard, in FIG. 12, a description will be given of an example of using the transducer 73 in FIG. 9. As shown in FIG. 12, the transducer 73 is connected to transmitting/receiving means 82 having transmitting means 12 and receiving means 16. The transmitting/receiving means 82 has a transmitting/receiving separation switch 84 which connects to the transducer 73 by changing the transmitting means 12 and receiving means 16 in accordance with a command of the control means 24. Also, storage means (in the following, RAMs 86-1 to 86-7) for storing the signal strength of the ultrasonic waves transmitted from the sections P1 to P7 of the transducer 73 is provided for each section. Also, correction control means 88 for generating a correction command based on the signal strength read from the RAMs 86-1 to 86-7 and outputting the command to the control means 24 is provided. The correction command is a command to adjust an electromechanical coupling coefficient of each oscillation element (or for each section, or else for each group) based on the signal strength read out from the RAMs 86-1 to 86-7 to a setting value. Also, bias means 14 for applying bias voltages having predetermined values to the sections P1 to P7 of the transducer 73 is disposed. In this regard, a digital-analog conversion means 90 for converting the drive signal from a digital signal to an analog signal is connected at the preceding stage of the transmitting means 12. Also, an analog-digital conversion means 92 for converting the reflection echo signal output from the transducer 73 from an analog signal to a digital signal is connected at the succeeding stage of the receiving means 16.

In the present embodiment, before starting ultrasonic imaging, the bias means 14 applies a common bias voltage g₀(n) to oscillation elements pertaining to each of the sections P1 to P7. By this, ultrasonic waves are transmitted from the oscillation elements pertaining to each of the sections P1 to P7. The strength of the signal of the transmitted ultrasonic wave is measured for each of the sections P1 to P7. The measured signal strength is stored in each of the RAMs 86-1 to 86-7 corresponding to each of the sections P1 to P7 (preliminary measurement process). The difference between the signal strength read out from the RAMs 86-1 to 86-7 and a predetermined setting value is obtained by the correction control means 88. A correction bias voltage to be the setting value of the electromechanical coupling coefficient for each of the sections P1 to P7 is calculated based on the obtained difference. The calculated correction bias is output from the correction control means 88 to the control means 24 (correction process). The control means 24 outputs a command to the bias means 14 based on the output correction bias voltage. The bias means 14 applies the correction bias voltages to each of the sections P1 to P7 in accordance with the command from the control means 24.

A detailed description will be given of the control of the correction control means 88. It is assumed that the electromechanical coupling coefficient of each of the sections P1 to P7 is f(n). When a drive signal with an amplitude of “1” is input into each of the sections P1 to P7, the ultrasonic signal S transmitted for each of the sections P1 to P7 is represented by α×f(n). In this regard, n is the number of the section and α is a predetermined coefficient.

If the electromechanical coupling coefficients of the individual the sections P1 to P7 are the same, the ultrasonic signals S transmitted for each of the sections P1 to P7 become the same. However, if the electromechanical coupling coefficients of the individual sections P1 to P7 are different (FIG. 13A), the ultrasonic signals S transmitted become different. In that case, the ultrasonic waves transmitted from the individual sections P1 to P7 are sometimes intensified with each other at positions other than a focal point because of the differences of the signal strength of the individual ultrasonic signals S. Accordingly, unnecessary responses arise, and thus artifacts, etc., may sometimes occur in the ultrasound beams.

On this point, in the present embodiment, the correction bias voltage g(n) for making uniform the ultrasonic signals of each of the sections P1 to P7 by the correction control means 88 is calculated as the expression 1.
g(n)=g₀(n)/{α×f(n)} (Expression 1)

As is understood from the expression 1, the bias voltage is weighted in accordance with the value of the ultrasonic signal S of each of the sections P1 to P7 (FIG. 13B), the electromechanical coupling coefficients of individual sections P1 to P7 are corrected so as to be equivalent to the case of a uniform coefficient (FIG. 13C).

According to the present embodiment, when oscillation elements and sections P1 to P7 are formed in an transducer, if variations arise in the electromechanical coupling coefficients of the sections P1 to P7 caused by the formation process of the oscillation elements and sections, the bias voltages to be applied to the individual sections P1 to P7 are corrected in accordance with those variations. Thus, it becomes equivalent to the case where the electromechanical coupling coefficients of the individual sections P1 to P7 are uniform. This produces results in which the ultrasonic waves transmitted from individual sections P1 to P7 increase the strength at the focal point and decrease the strength at the other points, and thereby making it possible to form good ultrasonic beams.

In the present embodiment, a description will be given of the example in which bias voltages to be applied to the individual sections P1 to P7 are corrected based on the variations of the electromechanical coupling coefficients for each of the sections P1 to P7. However, the corrections may be performed for each transducer or for each oscillation element. Also, the present embodiment can be appropriately combined with the first to the fifth embodiments and the variations thereof.

### Seventh Embodiment

A description will be given of a seventh embodiment of an ultrasonic probe to which the present invention is applied and an ultrasonic imaging apparatus. The present embodiment is different from the sixth embodiment in the point that the variations due to the transmitting/receiving circuit are corrected. The description of the same portion as that of the sixth embodiment is omitted, and a description will be given on the different points.

In the present embodiment, the RAMs 86-1 to 86-7 in FIG. 12 stores information produced by adding variations of the signal caused by the transmitting means 12, the receiving means 16, and the transmitting/receiving separation switch 84 to the electromechanical coupling coefficients.

For example, assume that the output signal of the transmitting means 12 is T(n) when a drive signal with an amplitude of “1” is input into the transmitting means 12. Also, assume that the output signal of the transmitting/receiving separation switch 84 is TR-t(n) when a drive signal with an amplitude of “1” is input into the transmitting/receiving separation switch 84. In this case, the ultrasonic signal S_{T} emitted from each of the sections P1 to P7 is represented as the expression 2. Accordingly, the correction control means 88 calculates the correction bias signal gₜ(n) to be applied to each of the sections P1 to P7 as the expression 3. As is understood from the expression 3, the correction is performed equivalently to the case where there are no signal variations which are caused by the transmitting system circuit and which influence on the ultrasonic wave transmitted from each of the sections P1 to P7. By this means, it is possible to decrease the artifact caused by the ultrasound image so as to improve the S/N of the ultrasound image.
S_{T}=T(n)×TR-t(n)×(α×f(n)) (Expression 2)
gₜ(n)=g₀(n)/S_{T} (Expression 3)

Also, assume that the output signal of the transmitting/receiving separation switch 84 is TR-r(n) when a reflection echo signal with an amplitude of “1” is input into the transmitting/receiving separation switch 84. Also, assume that the output signal of the receiving means 16 is R(n) when a reflection echo signal with an amplitude of “1” is input into the receiving means 16. In this case, the reflection echo signal S_{R} output from the receiving means 16 for each of the sections P1 to P7 is represented as the expression 4. Accordingly, the correction control means 88 calculates the correction bias signal gᵣ(n) to be applied to each of the sections P1 to P7 as the expression 5. By this means, the correction is performed equivalently to the case where there are no signal variations which are caused by the receiving system circuit and which influence on the reflection echo signal output from each of the sections P1 to P7. By this means, it is possible to decrease the artifact caused by the ultrasound image so as to improve the S/N of the ultrasound image.
S_{R}=TR−r(n)×R(n)×(α×f(n)) (Expression 4)
gᵣ(n)=g₀(n)/S_{R} (Expression 5)

According to the present embodiment, the bias signal gₜ(n) is applied to each of the sections P1 to P7 when the ultrasound beams are transmitted. When ultrasound beams are received, the bias signal is changed to the bias signal gᵣ(n) to be applied. Thus it is possible to correct the variations of the ultrasonic signals caused by the transmitting/receiving separation switch 84, the transmitting means 12, and the receiving means 16 in addition to the variations of the electromechanical coupling coefficients. Accordingly, it is possible to decrease the artifact caused by the ultrasound image so as to improve the S/N of the ultrasound image.

In summary, the present embodiment has a preliminary measurement process in which the direct-current bias g₀(n) is applied to the oscillation elements for each of the sections P1 to P7 and the electromechanical coupling coefficients of individual sections P1 to P7 are measured. Also, the present embodiment has a correction process in which the value of the direct-current bias g₀(n) is corrected to gᵣ(n) based on the measured electromechanical coupling coefficients. By applying the bias with changing the direct-current bias gₜ(n) applied to the oscillation elements when the oscillation elements transmit the ultrasonic waves, and the direct-current bias gᵣ(n) applied to the oscillation elements when the oscillation elements receive the waves, it is possible to correct the signal variations of the transmitting system circuit and the signal variations of the receiving system, respectively. In this regard, the value of the direct-current bias gₜ(n) may be different from the direct-current bias gᵣ(n).

In the present embodiment, a description has been given of the example in which bias voltages to be applied to the individual sections P1 to P7 are corrected based on the variations of the electromechanical coupling coefficients for the individual sections P1 to P7. However, the corrections may be performed for each transducer or for each oscillation element. Also, the present embodiment can be appropriately combined with the first to the fifth embodiments and the variations thereof.

The present invention has been described based on the embodiments. However, the present invention is not limited to these. For example, in FIG. 7, an example in which ultrasonic waves which are formed symmetrically in the minor-axis direction with the central position of the ultrasonic aperture as a center by weighting for each section the values of the bias voltage to be applied to the sections P1 to P3 is shown. However, the ultrasound beams may be biased by controlling the value of the bias voltage for each section. In summary, the ultrasound beams transmitted and received by the ultrasonic probe may be biased by dividing a plurality of oscillation elements into a plurality of sections in the minor-axis direction and by weighting the value of the direct-current bias applied to each oscillation element for each group asymmetrically with the central position of the ultrasonic aperture as the center. In this regard, the same is also applied for the major-axis direction.

Also, in FIG. 4, it's shown that one example of an oscillation element made of the material including a semiconductor compound. However, it is also possible to form an oscillation element from an electostrictive material. For the electostrictive material, a porcelain composition having a phase-transition temperature to a ferroelectric, which is relatively near room temperature, in a relaxation ferroelectric, such as Pb(Mg_{1/3}Nb_{2/3})O₃—PbTiO₃ series solid solution ceramics, and a composite material produced by dividing the porcelain plate into many minute columns vertically and horizontally and filling the division gaps with resin, etc., may be used. In summary, the oscillation element may be formed by a material having an electromechanical coupling coefficient which changes by the value of the applied bias voltage.

1. An ultrasonic probe including a plurality of transducers in an array for converting drive signals into ultrasonic waves to transmit the waves to an object to be inspected and converting the waves into electrical signals to receive ultrasonic waves generated from the object, wherein
each of the transducers comprises a plurality of oscillation elements, each of the oscillation elements has a characteristic of changing an electromechanical coupling coefficient in accordance with strength of a direct-current bias applied by being superposed on the drive signal, and an electrode of each of the transducers is connected to a terminal provided with the drive signal.

2. The ultrasonic probe according to claim 1, wherein the plurality of oscillation elements are divided into a plurality of groups, and the electrode of each of the oscillation elements pertaining to a same group are commonly connected.

3. The ultrasonic probe according to claim 1, wherein the plurality of oscillation elements are divided into a plurality of groups in a minor-axis direction, and the electrode of each of the oscillation elements pertaining to a same group are commonly connected.

4. The ultrasonic probe according to claim 1, wherein the plurality of oscillation elements are divided into a plurality of groups in a major-axis direction, and the electrode of each of the oscillation elements pertaining to a same group are commonly connected.

5. The ultrasonic probe according to claim 1, wherein the plurality of oscillation elements are formed at equal intervals, the oscillation elements are divided into a plurality of groups having an equal number of the oscillation elements, and the electrode of each of the oscillation elements pertaining to a same group are commonly connected.

6. The ultrasonic probe according to claim 1, wherein the plurality of oscillation elements are divided into a plurality of groups, a number of the oscillation elements pertaining to each of the divided groups increases for each group as the element gets closer a center of an ultrasonic aperture, and the electrode of each of the oscillation elements pertaining to a same group are commonly connected.

7. The ultrasonic probe according to claim 1, wherein the terminal is connected to a power source through switching means.

8. The ultrasonic probe according to claim 1, wherein the oscillation elements are formed by a material including a semiconductor compound.

9. An ultrasonic imaging apparatus comprising: an ultrasonic probe according to claim 1; transmitting means for supplying drive signals to the oscillation elements of the ultrasonic probe; receiving means for processing electrical signals output from the oscillation elements; and image processing means for reconstructing an ultrasound image based on signals output from the receiving means; wherein bias means applying a direct-current bias on the oscillation elements by superposing the bias on the drive signal is connected to electrodes of the oscillation elements through the terminal.

10. The ultrasonic imaging apparatus according to claim 9, wherein the bias means includes a direct-current power source, distribution means for dividing a direct-current bias provided from the direct-current power source, and switching means for applying each direct-current bias supplied from the distribution means to electrodes of the oscillation elements in accordance with a control command through the terminal.

11. The ultrasonic imaging apparatus according to claim 9, wherein the plurality of oscillation elements are divided into a plurality of groups, and the bias means applies a direct-current bias having different strength for each of the groups to each of the oscillation elements.

12. The ultrasonic imaging apparatus according to claim 9, wherein the plurality of oscillation elements are divided into a plurality of groups in a minor-axis direction, and the bias means applies a direct-current bias having different strength for each of the groups to each of the oscillation elements.

13. The ultrasonic imaging apparatus according to claim 9, wherein the plurality of oscillation elements are divided into a plurality of groups in a major-axis direction, and the bias means applies a direct-current bias having different strength for each of the groups to each of the oscillation elements.

14. The ultrasonic imaging apparatus according to claim 9, wherein the plurality of oscillation elements are divided into a plurality of groups, and the bias means applies a direct-current bias increasing for each group as the elements gets closer a center of an ultrasonic aperture.

15. The ultrasonic imaging apparatus according to claim 9, wherein the bias means applies a direct-current bias to each oscillation element such that an electromechanical coupling coefficient of each of the oscillation elements increases as the element gets closer a center of a minor-axis direction.

16. The ultrasonic imaging apparatus according to claim 9, wherein the plurality of oscillation elements are divided into a plurality of groups, and the bias means selects the oscillation element to which a direct-current bias is applied for each group in accordance with a distance from the ultrasonic probe to an imaging portion.

17. The ultrasonic imaging apparatus according to claim 9, further comprising: storage means for storing signal strength of an ultrasonic wave transmitted from each of the oscillation elements and correction control means for generating a command to correct an electromechanical coupling coefficient of each of the oscillation elements based on the signal strength to a setting value, wherein the bias means applies a direct-current bias corrected based on the correction command to each of the oscillation elements.

18. The ultrasonic imaging apparatus according to claim 9, wherein the bias means alternatively applies a direct-current bias applied to each of the oscillation elements when an ultrasonic wave is transmitted from each of the oscillation elements to the object, or applies a direct-current bias to each of the oscillation elements when ultrasonic waves generated from the object are received by each of the oscillation elements.

19. The ultrasonic imaging apparatus according to claim 9, wherein the plurality of oscillation elements are divided into a plurality of groups, and the bias means applies a direct-current bias having weight for each group symmetrically with respect to a center of an ultrasonic aperture in a minor-axis direction or in a major-axis direction to each of the oscillation elements.

20. The ultrasonic imaging apparatus according to claim 9, wherein the plurality of oscillation elements are divided into a plurality of groups, and the bias means applies a direct-current bias having weight for each group asymmetrically with respect to a center of an ultrasonic aperture in a minor-axis direction or in a major-axis direction to each of the oscillation elements.

21. A method of ultrasonic imaging comprising:
a step for applying a direct-current bias to a plurality of oscillation elements possessed by each transducer arrayed in an ultrasonic probe and changing an electromechanical coupling coefficient of each of the oscillation elements to a setting value;
a step for supplying a drive signal to each of the oscillation elements by superposing the drive signal on the direct-current bias and transmitting an ultrasonic wave to an object to be inspected from each of the oscillation elements; and
a step for receiving an ultrasonic wave generated by the object by each of the oscillation elements to convert the wave into an electrical signal and reconstructing an ultrasound image based on the converted electrical signal.
